(19) United States
(12) Patent Application Publication
(10) Pub. No.: US 2021/0133981 A1
(43) Pub. Date: May 6, 2021

(54) BIOLOGY DRIVEN APPROACH TO IMAGE SEGMENTATION USING SUPERVISED DEEP LEARNING-BASED SEGMENTATION

(71) Applicant: Allen Institute, Seattle, WA (US)

(72) Inventors: Jianxu Chen, Shoreline, WA (US); Susanne Marie Rafelski, Seattle, WA (US)

(21) Appl. No.: 17/085,509

(22) Filed: Oct. 30, 2020

Related U.S. Application Data
(60) Provisional application No. 62/928,144, filed on Oct. 30, 2019.

(51) Int. Cl. G06T 7/149; G06T 7/11; G06T 7/00; G16H 30/40; G16H 30/20; G06N 3/08

(52) U.S. Cl. CPC G06T 7/149; G06T 7/11; G06T 7/0012; G06T 2207/10056; G16H 30/20; G06N 3/08; G16H 30/40

(57) ABSTRACT

A segmentation machine learning model is described that has trained to predict segmentation for images captured in a first manner using training observations that each pair an image of a scene captured in the first manner with a segmentation of an image of the same scene captured in a second manner distinct from the first manner, where the segmentation of the image of the same scene captured in the second manner was produced by applying to the image of the same scene captured in the second manner a model for segmenting images captured in the second manner. The model can be applied to a distinguished image captured in the first manner to predict a segmentation of the distinguished image.

### CROSS-REFERENCE TO RELATED APPLICATIONS

This Application claims the benefit of provisional U.S. Application No. 62/928,144, filed Oct. 30, 2019 and entitled “BIOLOGY DRIVEN APPROACH TO IMAGE SEGMENTATION USING SUPERVISED DEEP LEARNING-BASED SEGMENTATION,” which is hereby incorporated by reference in its entirety, and is related to U.S. Patent Application No. 62/752,878, filed Oct. 30, 2018 and entitled “SEGMENTING 3D INTRACELLULAR STRUCTURES IN MICROSCOPY IMAGES USING AN ITERATIVE DEEP LEARNING WORKFLOW THAT INCORPORATES HUMAN CONTRIBUTIONS”; U.S. Patent Application No. 62/775,775, filed Dec. 5, 2018, 2018 and entitled “SEGMENTING 3D INTRACELLULAR STRUCTURES IN MICROSCOPY IMAGES USING AN ITERATIVE DEEP LEARNING WORKFLOW THAT INCORPORATES HUMAN CONTRIBUTIONS”; U.S. Patent Application No. 16/669,089, filed Oct. 30, 2019 entitled “SEGMENTING 3D INTRACELLULAR STRUCTURES IN MICROSCOPY IMAGES USING AN ITERATIVE DEEP LEARNING WORKFLOW THAT INCORPORATES HUMAN CONTRIBUTIONS”; U.S. PCT Application No. PCT/US19/58894, filed Oct. 30, 2019 and entitled “SEGMENTING 3D INTRACELLULAR STRUCTURES IN MICROSCOPY IMAGES USING AN ITERATIVE DEEP LEARNING WORKFLOW THAT INCORPORATES HUMAN CONTRIBUTIONS,” and U.S. PCT Application No. PCT/US18/45840, filed Aug. 8, 2018 and entitled “SYSTEMS, DEVICES, AND METHODS FOR IMAGE PROCESSING TO GENERATE AN IMAGE HAVING PREDICTIVE TAGGING,” each of which is hereby incorporated by reference in its entirety.

In cases where the present application conflicts with a document incorporated by reference, the present application controls.

### BACKGROUND

Segmentation refers to the identification of the borders of structures appearing in images, such as the borders of cells and cell nuclei appearing in 3D microscope images.

Deep neural networks have been used to perform segmentation in microscopy images, and have achieved success for some problems too difficult to tackle by manual or procedural image processing techniques. In these conventional uses of deep neural networks, the deep neural network is trained by either a supervised or an unsupervised approach. The unsupervised learning approach can be a good way to roughly detect the signal without requiring a pixelwise segmentation target, but with very restricted applicability. Currently, supervised learning is the approach that tends to achieve the most accurate segmentation. Most supervised learning strategies for deep neural networks require large sets of ground truth segmentation data for training. (Here, “ground truth” is defined as the target segmentation of the input image, which does not claim to represent any real truth). Currently, manually annotating the pixels/voxels in images is commonly used for ground truth collection.

### BRIEF DESCRIPTION OF DRAWINGS

The patent or application file contains at least one drawing executed in color. Copies of this patent or patent application publication with color drawing(s) will be provided by the Office upon request and payment of the necessary fee.

FIG. 1 is a block diagram showing some of the components typically incorporated in at least some of the computer systems and other devices on which the facility operates.

FIG. 2 is a flow diagram showing a process performed by the facility in some embodiments in order to establish a segmentation model for a primary assay.

FIG. 3 discussed below, which shows a multi-channel 3D microscopy image with both DNA dye and Lamin B1 of the same field of view (FOV).

FIG. 4 is a dataflow diagram showing an overview of workflow used by the facility in some embodiments to build a deep neural network for accurate semantic segmentation—of the entire field of view, not individually detected objects—of nuclei/mitotic DNA from DNA dye via two secondary assays for training.

FIG. 5 is a dataflow diagram showing the workflow used by the facility in some embodiments to build a deep neural network for accurate semantic segmentation of cell membrane via a cell membrane dye and a secondary assay for training.

FIG. 6 is a dataflow diagram showing a process used by the facility in some embodiments to convert the prediction outputs from the nucleus/mitotic DNA segmentation model, cell membrane segmentation model, seeding model and the pair detection model into instance segmentations of all cells and nuclei/mitotic DNA in an image the facility performs the dataflow on a two-channel image containing a DNA dye component 610 and a membrane dye component 616, and proceeds as follows with different operation types shown based on legend 601.

FIG. 7 is an image diagram showing qualitative nuclear segmentation accuracy at the voxel level.

FIG. 8 is an image diagram showing a sample result of evaluating the performance of cell instant segmentation at the voxel level using a held-out subset of the CAAX data set.

### DETAILED DESCRIPTION

### 1. Overview

The inventors have recognized that manual annotation is very subjective and may introduce bias. Also, the annotation of 3D images is extremely time-consuming and the annotated shape tends to lack spatial smoothness, especially when the shape has complex morphology.

To address this issue, the Allen Cell Structure Segmenter (referred to as “the Segmenter” hereafter) operates using iterative deep learning, where automatic segmentations are first obtained and then curated by sorting or merging (instead of annotating the segmentation target voxels in 3D). The curated results are then used to train a deep neural network model for segmentation. Such cycles of “automatic segmentation, curation, training” can start from a few training images and repeat with more iterations to gradually enlarge the size of the training data and gradually improve the robustness and accuracy of segmentation. The iterative deep learning concept greatly reduces the ground truth collection effort and the potential bias arising from human annotation, as well as the size of the ground truth data required to start model training. Iterative deep learning is especially useful for generating segmentation models that are very robust to variations in large image data sets such that one model can be applied to an entire, large dataset even with e.g., day to day biological variation or slight variations in microscopy settings, etc. The Segmenter is described by Chen J, Ding L, Viana M P, Hendershott M C, Yang R, Mueller I A, Rafelski S M, The Allen Cell Structure Segmenter: a new open source toolkit for segmenting 3D intracellular structures in fluorescence microscopy image, bioRxiv, 2018 Jan. 1:491035, which is hereby incorporated by reference in its entirety.

In connection with the Segmenter, the inventors have conceived and reduced to practice a training assay approach in which biological experiments and computational algorithms for performing segmentation on behalf of those biological experiments are co-designed to produce more biologically correct segmentations (based on the segmentation target). The iterative deep learning workflow relies on starting with an accurate preliminary segmentation result. However, often a biological image-based assay is constrained by imaging requirements of the experimental system (such as requirement for reduced light in live cell imaging), and thus generates assay images that may be difficult to use directly to create these accurate preliminary segmentations.

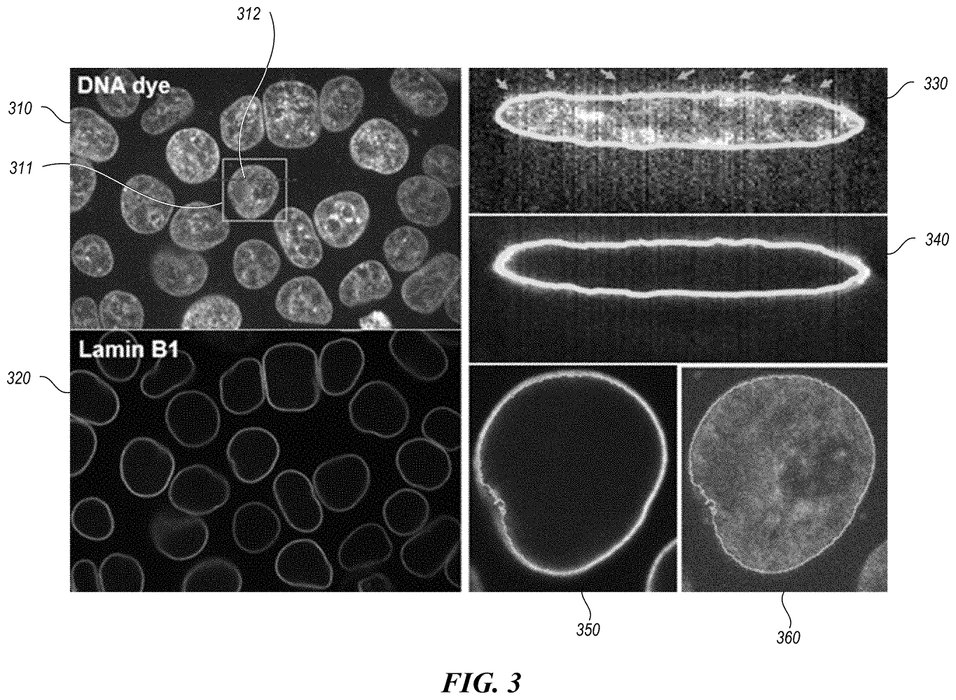

Considering the nature of this iterative deep learning workflow, the training assay approach ties a secondary assay more amenable to accurate segmentation to the primary image-based assay, and thus permits the training of a segmentation model that achieves the accuracy as in the secondary assay when run on the primary image-based assay. An example of this concept appears in FIG. 3 discussed below, which shows a multi-channel 3D microscopy image with both DNA dye and Lamin B1 of the same field of view (FOV). The inventors observe that it is hard to accurately detect the boundaries of interphase nuclei only based on DNA dye, especially in the Z direction due to limitations in microscopy optics including the diffraction of light. However, the nuclear envelope can be visualized via Lamin B1, which creates a “shell” around the nucleus and thus permits a more biologically accurate detection of the nuclear boundary, especially in the Z direction. Such boundaries obtained from this separate, co-designed, image-based assay can then be used to train a deep learning model for accurate segmentation of interphase nuclei from DNA dye and applied to DNA dye images from other assays.

The training assay approach is a general computation-experiment co-design concept applicable to many challenging segmentation problems discussed further below. The training assay approach can be used in some embodiments to perform accurate 3D semantic segmentation (foreground/background in entire field of view, without object detection) of nuclei (and DNA during mitosis) and cell boundaries in high resolution/magnification 3D spinning disk confocal microscopy images of colonies human induced pluripotent stem cells (hiPSCs). In these images the nuclei and the cell membranes are visualized by DNA dye (NucBlue Live, Hoechst) and membrane dye (Cell Mask Deep Red), respectively. The training assay approach then transforms the semantic segmentation into accurate instance segmentation of cells and nuclei (each cell and nucleus detected as a separate object) to demonstrate the required next steps to achieve the target result, which is a segmentation of all cells and nuclei in an image. This general approach and steps in this workflow generalize to other cell types and microscope image modalities. The joint iterative deep learning and training assay approach described herein permits researchers to segment many cells and nuclei in the inventors' 3D microscopy images, a fundamental challenge to performing image-based single cell analysis at scale. The images used here are from the Allen Cell Image Data Collection (allencell.org).

Thus, the inventors have conceived and reduced to practice a software and/or hardware facility for performing improved image segmentation (“the facility”), which in various embodiments incorporates versions of the segmenter and/or the training assay approach.

### 2. Hardware

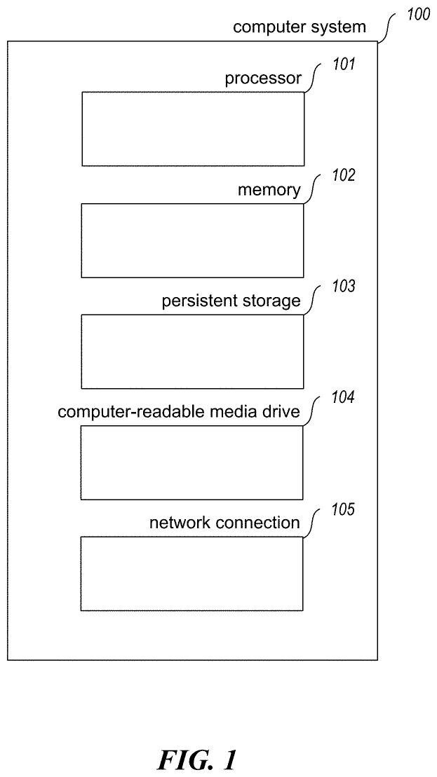

FIG. 1 is a block diagram showing some of the components typically incorporated in at least some of the computer systems and other devices on which the facility operates. In various embodiments, these computer systems and other devices 100 can include server computer systems, desktop computer systems, laptop computer systems, cloud computing platforms for virtual machines in other configurations, netbooks, tablets, mobile phones, personal digital assistants, televisions, cameras, automobile computers, electronic media players, smart watches and other wearable computing devices, etc. In various embodiments, the computer systems and devices include one or more of each of the following: a central processing unit (“CPU”), graphics processing unit (“GPU”), or other processor 101 for executing computer programs; a computer memory 102 for storing programs and data while they are being used, including the facility and associated data, an operating system including a kernel, and device drivers; a persistent storage device 03, such as a hard drive or flash drive for persistently storing programs and data; a computer-readable media drive 104, such as a floppy, CD-ROM, or DVD drive, for reading programs and data stored on a computer-readable medium; and a network connection 105 for connecting the computer system to other computer systems to send and/or receive data, such as via the Internet or another network and its networking hardware, such as switches, routers, repeaters, electrical cables and optical fibers, light emitters and receivers, radio transmitters and receivers, and the like. While computer systems configured as described above are typically used to support the operation of the facility, those skilled in the art will appreciate that the facility may be implemented using devices of various types and configurations, and having various components. In various embodiments, the computing system or other device also has some or all of the following hardware components: a display usable to present visual information to a user; one or more touchscreen sensors arranged with the display to detect a user's touch interactions with the display; a pointing device such as a mouse, trackpad, or trackball that can be used by a user to perform gestures and/or interactions with displayed visual content; an image sensor, light sensor, and/or proximity sensor that can be used to detect a user's gestures performed nearby the device; and a battery or other self-contained source of electrical energy that enables the device to operate while in motion, or while otherwise not connected to an external source of electrical energy.

### 3. Process

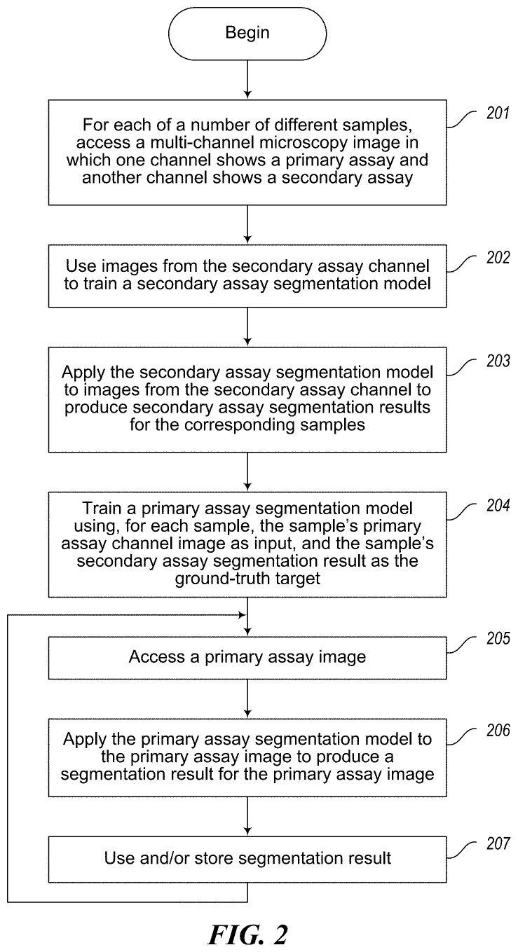

FIG. 2 is a flow diagram showing a process performed by the facility in some embodiments in order to establish a segmentation model for a primary assay. In act 201, the facility accesses a multi-channel microscopy image of each of a number of different biological samples. In each multi-channel microscopy image, one channel shows a primary assay—that is, an image of the sample captured using a first imaging technique—as well as another channel showing a secondary assay—an image of the sample captured using a second imaging technique. In act 202, the facility uses some or all of the images from the secondary assay channel to train a secondary assay segmentation model. The facility's training of the secondary assay segmentation model is discussed in greater detail below. In some embodiments (not shown), at least some of the images used by the facility to train the secondary assay segmentation model are not paired with primary assay images of the same sample.

In act 203, the facility applies the secondary assay segmentation model trained in act 202 to images from the secondary assay channel to produce secondary assay segmentation results for the corresponding samples. In act 204, the facility trains a primary assay segmentation model. In doing so, the facility uses, for each sample, (1) the sample's primary assay channel image as training input, and (2) the sample's secondary assay segmentation result determined in act 203 as the ground-truth target for that input. In act 205, the facility accesses a primary assay image—that is, any image captured using the first image capture technique. In act 206, the facility applies the primary assay segmentation model trained in act 204 to the primary assay image accessed in act 205 to produce a segmentation result for that primary assay image. In 207, the facility uses, displays, and/or stores the segmentation result obtained in act 206. After act 207, the facility continues in act 205 to access the next primary assay image for segmentation.

Those skilled in the art will appreciate that the acts shown in FIG. 2 may be altered in a variety of ways. For example, the order of the acts may be rearranged, some acts may be performed in parallel; shown acts may be omitted, or other acts may be included; a shown act may be divided into subacts, or multiple shown acts may be combined into a single act; etc.

### 4. Results

4.1 Training Assays for Semantic Segmentation of Nuclei/Mitotic DNA

Note: “Nuclei” is used herein to refer both nuclei in interphase cells and reforming nuclei in two daughter cells in the telophase and cytokinesis stages of mitosis. Mitotic DNA is used to refer the “nuclei” in the prophase, metaphase, and anaphase stages.

In 3D fluorescent spinning disk confocal microscopy, a cellular structure imaged as a solid shape, like the DNA representing the filled nucleus, often has blurry boundaries due to the diffraction of light, which is especially limiting in the axial Z direction. As a consequence, locating an accurate and consistent boundary of a filled fluorescent nucleus merely from the fluorescent images is challenging. For this reason, in some embodiments, the facility leverages specialized biological experiments to obtain a preliminary nuclear segmentation with a more biologically correct boundary. Starting from the preliminary segmentation, the facility then uses iterative deep learning to build a deep neural network model that can segment nuclei from 3D microscopy images with more accurate boundaries than are visible in the assay data the model is applied to.

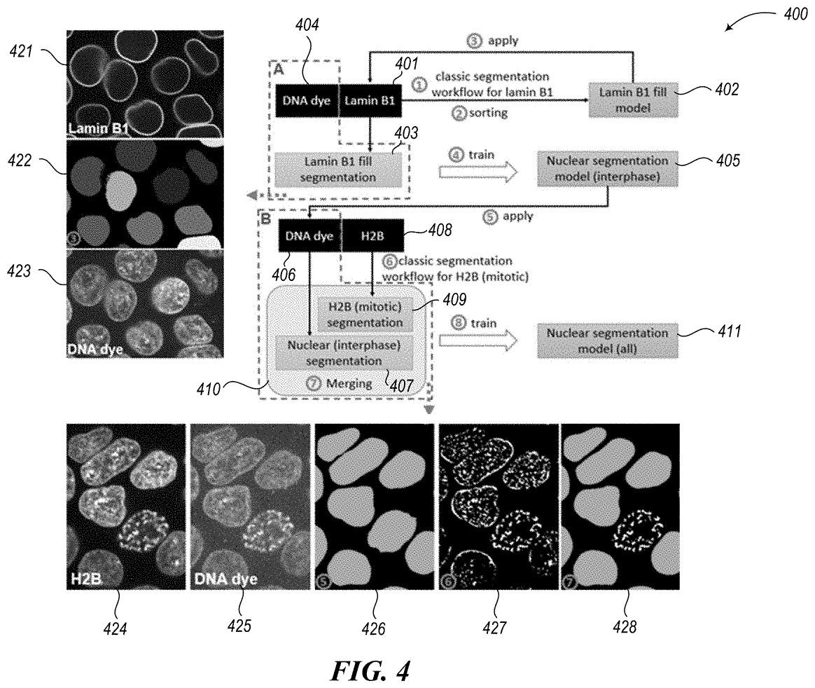

Data from specialized co-designed experimental assays to detect nuclei and mitotic DNA: The objective here is to accurately identify the nuclei of interphase cells and the mitotic DNA during mitosis (representing the “nucleus” during nuclear envelope break-down). In some embodiments, the training assay approach applied by the facility uses two distinct co-designed experimental assays as secondary assays: (1) mEGFP-tagged Lamin B1 (labels the nuclear envelope) for interphase nuclei and (2) mEGFP-tagged H2B (labels a subset of DNA histones) from the Allen Cell Image Data Collection. In each image, the cells in the same FOV are imaged in four different channels: DNA dye, mEGFP-tagged Lamin B1 (or mEGFP-tagged H2B), plasma membrane dye, and bright-field transmitted light. Lamin B1 labels a subset of the nuclear lamina located just inside the inner nuclear envelope. For hiPS cells during interphase, the segmentation equivalent of a filled Lamin B1 shell represents the nucleus; these are shown in FIGS. 3 and 4 discussed below. During mitosis, the Lamin B1 signal re-localizes to other regions of the cell as the nuclear envelope partially breaks down. During mitosis, the facility's segmentation target is the condensed mitotic DNA, which then decondenses at the end of mitosis as the nuclei reform. Mitotic DNA is visible via the DNA dye but has much more consistent and higher image quality when imaged via endogenously tagged mEGFP-tagged H2B, which the inventors thus use as a “cleaner” equivalent of mitotic DNA during mitosis.

FIG. 3 is an image diagram showing a sample image using DNA dye and Lamin B1. The diagram shows a DNA dye image 310 of the sample, as well as a Lamin B1 image 320 of the sample. Both of these images show the same scene: the same field-of-view for the same sample at the same level of magnification. Both of these two images are shown in the x-y dimension. It can be seen that the location, size, and shape of the nuclei shown in the DNA dye image are generally similar to those shown in the Lamin B1 image. A particular nucleus is shown in the DNA dye image by a yellow bounding box 311, and a red view line 312 is further shown through the nucleus in the DNA dye image. The diagram further includes an enlarged x-z slice 330 of the DNA image of the nucleus in the yellow box, cut along the red cut line. Image 340 is the corresponding slice of the Lamin B1 image. Image 350 is a zoomed x-y image of the nucleus in the Lamin B1 image, while image 360 is a zoomed x-y image of the nucleus from the DNA dye image. Images 340 and 350 show with a yellow line the result of automatically segmenting the Lamin B1 image for this nucleus. These same yellow segmentation lines are also overlaid on the zoomed images from the DNA dye image, images 330 and 360. This demonstrates the correspondence between the two assays in their portrayal of this nuclear boundary. Due to optical properties of the microscope including diffraction of light, especially along the Z direction, it is not straightforward to identify the exact boundary of the nucleus from the DNA dye image (see the green arrows). However, the nuclear envelope can be visualized via mEGFP-tagged Lamin B1, which creates a “shell” around the nucleus. This thus permits a more biologically accurate detection of the nuclear boundary, especially in the Z direction. This training assay-derived boundary can then be used to train a deep learning model to segment interphase nuclei from DNA dye but with the added accuracy from the Lamin B1 assay.

Main computational steps: FIG. 4 is a dataflow diagram showing an overview of workflow used by the facility in some embodiments to build a deep neural network for accurate semantic segmentation—of the entire field of view, not individually detected objects—of nuclei/mitotic DNA from DNA dye via two secondary assays for training. In a first portion of the dataflow, the facility begins with Lamin B1 images 401, such as Lamin B1 image 421. The facility uses these Lamin B1 images to train a Lamin B1 segmentation model 402. In some embodiments, the facility trains the Lamin B1 segmentation model by applying procedural segmentation techniques to a large number of Lamin B1 images; manually selecting the Lamin B1 images for which high-quality segmentation were produced by the procedural segmentation; and using these selected image/segmentation pairs to train a machine learning Lamin B1 segmentation model.

The facility applies this Lamin B1 segmentation model to the Lamin B1 channel of Lamin B1/DNA dye multi-channel images of a number of samples to automatically segment these Lamin B1 images. For example, image 422 shows the segmentation result produced for Lamin B1 image 421. These resulting Lam in B1 segmentation results 403 are matched with the corresponding DNA dye images 404 of the same samples, and together used to train a DNA dye nuclear segmentation model tuned for interphase nuclei 405. In particular, in each DNA dye image/Lamin B1 segmentation pair, the DNA dye image is used as the training input, and the Lamin B1 segmentation is used as the ground-truth target. For example, image 423 shows the DNA dye image that is paired with the Lamin B1 segmentation result 422 produced automatically from the Lamin B1 image 421 of the same sample.

In a second portion of the dataflow, multi-channel images of several samples are used that include a DNA dye channel and an H2B channel. These be the same or different samples compared to those used in the first portion of the dataflow. The facility first applies the nuclear segmentation model for interphase nuclei 405 to the DNA dye channel 406 of each image to produce a nuclear segmentation of the DNA dye image that is focused on interphase nuclei. The facility further performs segmentation on the H2B channel 408 to obtain a segmentation of each sample that is focused on accurately segmenting mitotic nuclei. For example, in some embodiments, the facility uses a classic segmentation work flow to obtain this H2B segmentation 409. For each sample, the facility merges the two segmentation results 410. In some embodiments, the facility performs the merging by directing the manual selection of mitotic nuclei from the H2B segmentation, and interphase nuclei in the DNA dye segmentation, then combining these selected nuclei in a single overall segmentation result. The facility then trains a comprehensive DNA dye segmentation model 411, for each sample using the DNA dye image as the training input, and the merged segmentation result as the ground-truth target. Segmentation model 411 can generate semantic segmentations of nuclei/mitotic DNA in all cell cycle stages.

Images 424-428 are example images corresponding to the second portion of the workflow. Image 424 is an example of an H2B channel 408; image 425 is an example of a DNA dye channel 406 for the same sample; image 426 is a segmentation result 407 produced by nuclear segmentation model 405 focused on interphase nuclei to image 425; image 427 is a segmentation result focused on mitotic nuclei 409 obtained by segmenting H2B 424; and image 428 is the merged segmentation result 410, containing the segmentation of interphase nuclei from segmentation result 426 and segmentation for mitotic nuclei from segmentation result 427.

4.2 Training Assay for Semantic Segmentation of the Cell Plasma Membrane

The signal from the plasma membrane dye used to label the boundaries of all the cells in this hiPSC image dataset can suffer from considerable photobleaching even using single z-stack acquisition via 3D spinning disk confocal. Cells are imaged from bottom to top, and thus the top of the cell membrane often shows very weak signal due to both dye labeling of a very thin membrane and this photobleaching problem. A weak signal at the top can make the accurate segmentation of the cell membrane via dye very challenging. In some embodiments, the training assay approach utilizes a specialized co-designed biological experiment to generate more accurate segmentations of the cell membrane top and impose this segmentation as the ground truth for training a deep neural network for the segmentation of cell membrane from membrane dye.

Data from a specialized co-designed experimental assay: The objective here is to accurately identify the boundaries of the cell in 3D via cell membrane dye. The co-designed secondary experimental assay in the training assay approach consists of using the CAAX cell line from the Allen Cell Image Data Collection. This cell line contains the membrane-targeting domain of K-Ras tagged with mTagRFP-T.

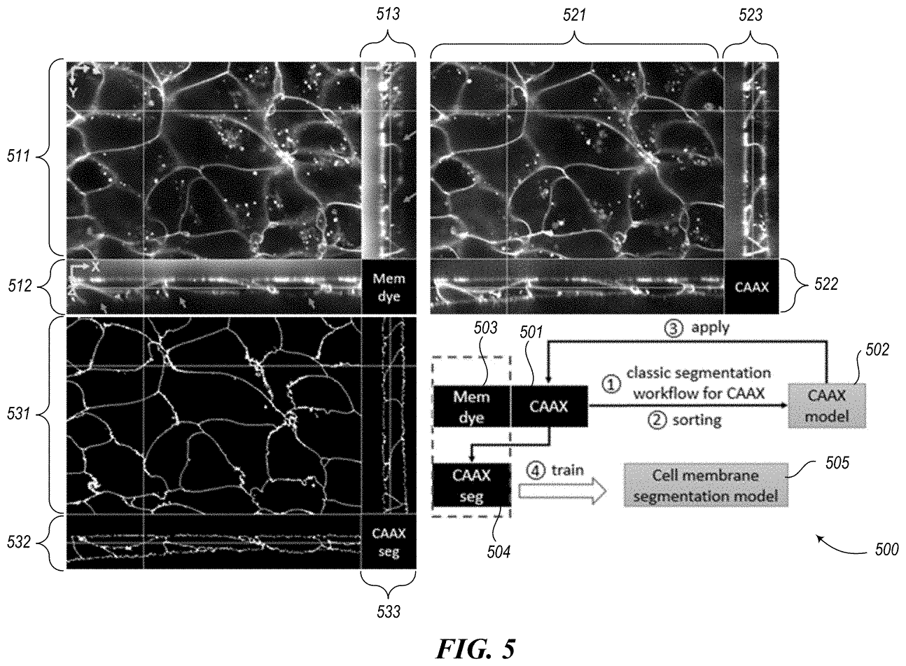

Main computational steps: FIG. 5 is a dataflow diagram showing the workflow used by the facility in some embodiments to build a deep neural network for accurate semantic segmentation of cell membrane via a cell membrane dye and a secondary assay for training.

For a particular sample, images 511-513 show a membrane dye image of the sample, while images 521-523 show an endogenously tagged cell membrane CAAX image of the sample. In particular, images 511 and 521 are in the x-y dimension, images 512 and 522 in the x-z dimension, and images 513 and 523 in the y-z dimension. The blue arrows in images 512 and 513 show that the cell boundaries are less distinct in the membrane dye image than the CAAX image, particularly in the z dimension; that is, the signal to noise ratio (SNR) of the cell membrane top in the CAAX channel is much stronger than in the membrane dye channel.

In the workflow 500, the facility begins with CAAX images 501, such as CAAX images 501, such as CAAX images 521-523. The facility uses the CAAX images to train a CAAX segmentation model 502. In some embodiments, the facility trains this CAAX segmentation model by applying procedural segmentation techniques to a large number of CAAX images; manually identifying the CAAX images for which the procedural segmentation techniques produce good results; and using these CAAX images and the corresponding segmentation results to train the machine leaning CAAX model 502. The facility then applies the trained CAAX model to the CAAX channel 501 of multi-channel images showing CAAX and membrane dye images 503 for the same samples. The application of the CAAX model to the CAAX images produces segmentation results 504 for the samples. These are used together with the membrane dye channel images 503 to train a cell membrane segmentation model 505. In particular, for each pair, the membrane dye image is used as model training input, and the CAAX segmentation is used as the ground-truth target. Images 531-533 show the segmentation 504 obtained by applying the CAAX segmentation model to the sample CAAX images 521-523.

4.3 3D Instance Segmentation of Cell and Nucleus

In addition to the two core deep learning models for semantic segmentation of cell membrane and nuclei/mitotic DNA, another two deep learning models are used by the facility in some embodiments for final instance segmentation to convert the binary field of view into individual cell and nuclei objects: a seeding model and a cell pair detection model.

Seeding model: The goal of the seeding model is to predict one single connected component for each individual nucleus from DNA dye images. The seeding model is similar to the nuclear segmentation model but predicts a slightly different nuclear mask. For cells in mitosis, the single connected component seed mask is built as the convex hull of the mitotic DNA segmentation mask of this cell. However, the cells in later anaphase and telophase/cytokinesis require different treatment because the mitotic DNA is separated into two regions of the cell or the two daughter nuclei begin to reform in the two daughter cells. These specific cells are permitted to and in fact should have two seeds per cell, one for each of the mitotic DNA/reforming nuclei that are pulled apart within a single cell. So, when building the seed mask for these cells, two convex hulls are computed for each of the mitotic DNA/reforming nuclei, instead of one. For cells in interphase, the single connected component seed mask is built as a “shrink” nuclear segmentation mask (implemented as a morphological erosion on the nuclear segmentation mask with a disk-shape structure element of radius 15 and taking the largest connected component in the eroded result). The “shrink mask” aims to encourage the seeding model to predict two separated seeds when two nuclei are tightly touching each other. In some embodiments, the facility uses the merging curator in Segmenter to modify the ground truth set generated for nuclear segmentation of two different types of cells (interphase and mitotic). About 600 mitotic cells are included. The new merged ground truth set is used to train the seeding model.

Pair detection model: It is helpful for each cell to have exactly one predicted seed from the seeding model in order to identify each instance. But, as discussed in the seeding model section, cells in late anaphase or telophase/cytokinesis naturally have two predicted seeds for each of the mitotic DNA/reforming nuclei that are pulled apart within a single cell. To address this issue, a pair detector based on FasterRCNN is trained (see Methods). FasterRCNN is described in Ren S, He K, Girshick R, Sun J, Faster r-cnn: Towards real-time object detection with region proposal networks, in Proceedings of Advances in Neural Information Processing Systems (pp. 91-99), which is hereby incorporated by reference in its entirety. The detector can automatically find the pair of mitotic DNA/reforming nuclei belonging to the same cell in late anaphase or telophase/cytokinesis and return a bounding box tightly encompassing each pair of mitotic DNA/reforming nuclei in late anaphase or telophase cytokinesis. Such bounding boxes are then used to locate the seed pairs.

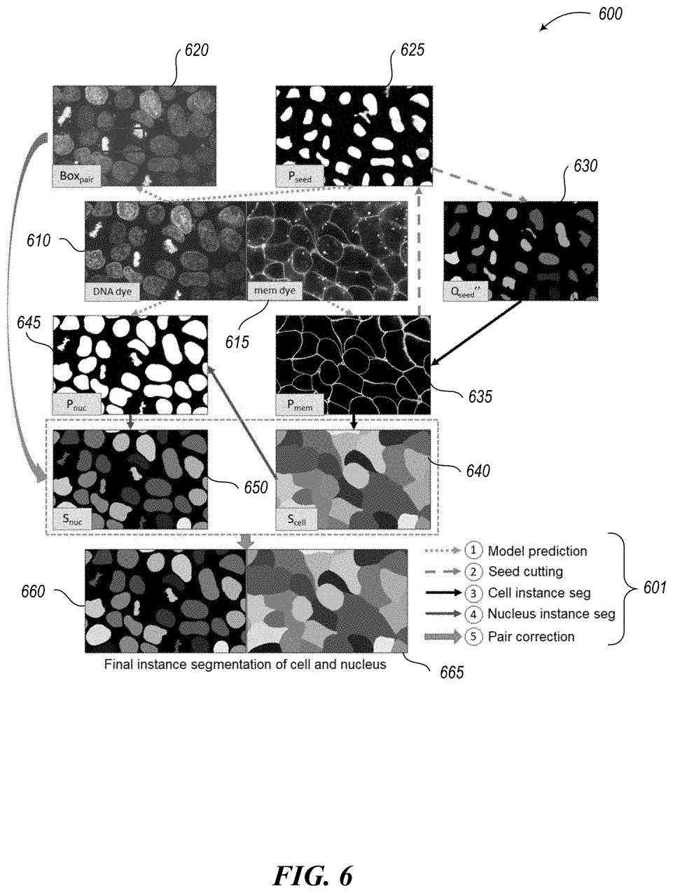

FIG. 6 is a dataflow diagram showing a process used by the facility in some embodiments to convert the prediction outputs from the nucleus/mitotic DNA segmentation model, cell membrane segmentation model, seeding model and the pair detection model into instance segmentations of all cells and nuclei/mitotic DNA in an image the facility performs the dataflow on a two-channel image containing a DNA dye component 610 and a membrane dye component 616, and proceeds as follows with different operation types shown based on legend 601.

1. Model Prediction:

The facility applies the mitotic segmentation model to the DNA dye image to obtain a nuclear segmentation P_{nuc}, shown as image 620. The facility applies the seeding model to the DNA dye channel in order to obtain the P_{seed} shrink nuclear segmentation mask 625 that tends to separate nuclei that adjoin one another. The facility applies the pair detection model to the DNA dye channel in order to obtain a boxₚₐᵢᵣ detection result 620 identifying sibling daughter nuclei that have recently separated. For example, the red rectangle in pair detection result 620 contains two sibling daughter nuclei identified by the pair detection model. The facility applies the cell membrane model to the membrane dye image to obtain a Pₘₑₘ membrane dye segmentation result 635.

2. Seed Cutting:

The facility applies empirically determined thresholds on Pₘₑₘ and P_{seed} (in some embodiments, thresholds of 0.7 and 0.9) to obtain the results Qₘₑₘ and Q_{seed}, respectively (not shown).

The facility performs cutting as follows: Q_{seed}′=Q_{seed}−Q_{seed}∩Qₘₑₘ.

The facility refines seeds: In Q_{seed}′, any connected components that have less than M voxels (M is set to 6,000 empirically) and are not adjacent to image borders are considered bad seeds and removed.

3. Cell Instance Segmentation:

The facility adds an auxiliary bottom seed: Starting from the bottom z-slice, the facility searches for the first z-slice in Qₘₑₘ with more than half of that z-slice belonging to cell membranes, say the result is Z₀. Then, all z-slices below Z₀ are considered an auxiliary bottom seed.

Add an auxiliary top seed: Starting from the top z-slice, the facility searches for the first z-slice in Qₘₑₘ with more than 50 voxels belonging to cell membranes to obtain a result Z₁. Then, all z-slices above Z₁ are considered an auxiliary top seed.

Where there are N connected components in Q_{seed}′, the final seed image Q_{seed}″ 630 is Q_{seed}′ with all z<max(2, Z₀) is set to N+1 and all z>=Z₁ set to N+2.

The facility performs seeded watershed on Pₘₑₘ to obtain S_{cell} 640. The seeds are a combination of all connected components in Q_{seed} and the extra auxiliary top and bottom seeds. (Watershed is performed with 26 connectivity in 3D with no watershed line.)

S_{cell}⁰ is the output from watershed after removing the connected components corresponding to the two auxiliary seeds. S_{cell}⁰ is refined near the bottom of the cells by finding a proper z-slice Z₂ near the bottom and copy the segmentation in Z₂ to z-slices from Z₀ to Z₂. This refined segmentation is S_{cell} 640.

4. Nucleus Instance Segmentation:

The facility applies an empirically determined threshold (in some embodiments, 0.5) on P_{nuc} and denote the result as Q_{nuc}.

The facility propagates the instance indices of each cell to nucleus: Q_{nuc}′=S_{cell}*Q_{nuc}.

In Q_{nuc}′, for each object (voxels with the same instance index), the facility performs minor refinement, either by filling holes and removing small objects, or by cleaning up small objects adjacent to the inner boundary of the same cell in S_{cell}.

5. Pair Correction:

The facility overlays all detected bounding boxes as in Boxₚₐᵢᵣ on S_{nuc} to extract the instance indices of each pair. If only one instance exists in the bounding box, this box is treated as false alarm and neglected; if more than two indices exist within the bounding box, the pair whose joint bounding box has highest overlap with the detected bounding box is selected.

In various embodiments, the facility verifies each pair either by a manual inspection or an automatic verification: checking if (1) the two corresponding nuclei have similar intensity, (2) both nuclei have more than 50% of the area enclosed within the detected bounding box (3) the sizes of two nuclei differ less than 30%, and (4) both nuclei have size smaller than estimated average size of nucleus in early interphase (e.g., G1 phase).

For each confirmed pair, the corresponding two instance indices are merged to the same index (in some embodiments, the smaller value of the two original indices) to obtain final nuclear segmentation 660. Final cell segmentation 665 is a copy of S_{cell} in which the merged instance indices are used.

4.4 Label-Free Integration

When applying instance segmentation algorithms on many of the types of images discussed here, a source of variation that may cause decreased segmentation performance is that the DNA dye or cell membrane dye may not strongly stain certain cells. This results in those cells being extremely dim, or even barely visible, in either the DNA or the cell membrane channel. Training on large data sets does not improve robustness in such scenarios due to the absence of the signal. While this problem is very rare (less than 1% of cells) due to the rigorous quality control of our cell lines and imaging pipeline, due to the large number of cells even this small percentage represents many cells (e.g., 200,000 cells in total means 1% is 2000 cells). In some embodiments, the facility avoids this type of error by using a predicted image tagging (“label-free”) technique. The label-free technique was originally designed to predict fluorescent images of different intracellular structures from bright field images in order to see different parts of a cell without any fluorescent marker. Here, the facility uses a similar approach to predict the segmentation mask from bright field images. Specifically, the facility trains the label-free model from bright field images to cell boundary segmentation (using the output from the cell membrane model), and from bright field images to nuclear segmentation (using output from DNA segmentation model) on a selected set of images (˜400 images). The two label-free models are denoted herein as LFnuc and LFmem. In some embodiments, LFnuc and LFmem are incorporated into the facility's operation as follows.

(1) The output of LFmem is combined with Pmem by weighted sum and used as the cell boundary prediction.

(2) The output of LFnuc is first binarized by an empirically determined threshold to obtain LFSegnuc. Then, LFSegnuc is processed similar to step 2 above to perform seed cutting. In the result image, any connected component that is not in Qseed (resp. Qnuc) is added in Qseed (resp. Qnuc).

4.5 Evaluation of Cell and Nuclear 3D Instance Segmentations

No “gold ground truth” is available to quantitatively evaluate the quality of the inventors' instance segmentation of cells and nuclei. The inventors therefore used an evaluation combining a voxel level qualitative assessment and an instance level quantitative assessment. To evaluate the quality of nuclear segmentation at the voxel level, the inventors use another specialized image-based biological assay, cells expressing mEGFP-tagged nucleoporin Nup153. Like Lamin B1, nucleoporin Nup153 forms a “shell” around the nucleus, as nucleoporin Nup153 is located on the nuclear side of the nuclear pore complex.

Nucleoporin Nup153 tags the nuclear pores and thus appears like densely packed puncta along the surface of the nucleus, instead of a continuous shell like Lam in B1. Puncta along the surface of the nucleus suffer even less from the diffraction of light compared to even the continuous shell of Lamin B1, and thus nucleoporin Nup153 data serves the purpose of evaluating the accuracy of the segmented nuclear boundary very well. If the nucleus segmentation is accurate, the boundary of the segmentation should reside near the center of those puncta along the nuclear boundary in 3D. The facility qualitatively evaluates nucleus segmentation at the voxel level using a specialized dataset of mEGFP-tagged nucleoporin Nup153 (labels the nuclear pores and forms a shell of puncta around the nucleus boundary).

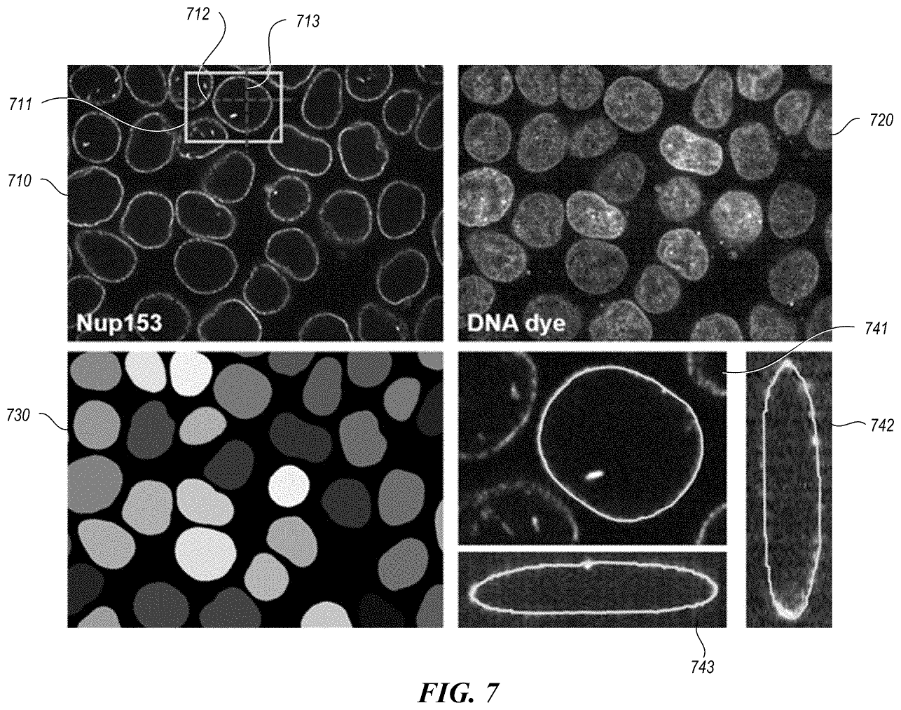

FIG. 7 is an image diagram showing qualitative nuclear segmentation accuracy at the voxel level. A multi-channel image includes a nucleoporin Nup153 channel 710, and a DNA dye channel 720. A particular nucleus is isolated in the nucleoporin Nup153 channel by yellow rectangle 711. Segmentation result 730 shows the instant segmentation of all nuclei from the DNA dye image. Image 741 is a zoomed-in version of the isolated image in the nucleoporin Nup153 image; image 742 shows a y-z slice of the nucleoporin Nup153 image along cut line 713; and image 743 shows an x-z slice of the isolated nucleus along cut line 712. In the zoomed-in images 741-743, a yellow line shows the imposition of the segmentation result produced from the DNA dye image. It can be seen that the segmented boundary is mostly along the center of the “shell” formed from nucleoporin Nup153, which confirms the biological correctness of the instance segmentation of nucleus even on an entirely different dataset than the Lamin B1 set the model was trained on.

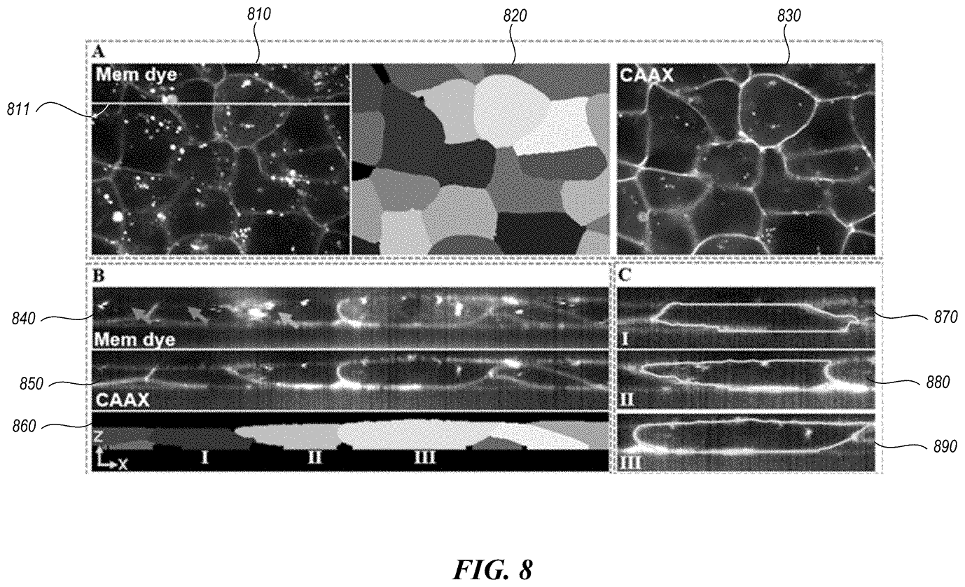

To qualitatively evaluate the performance of the cell instance segmentation at the voxel level, the inventors examined a small subset of the CAAX dataset held-out during the training. FIG. 8 is an image diagram showing a sample result of evaluating the performance of cell instant segmentation at the voxel level using a held-out subset of the CAAX data set. The diagram show, for a particular sample, an x-y slice 810 of a membrane dye channel, and x-y slice 830 of a CAAX channel. A segmentation result 820 produced by applying the facility's cell membrane segmentation model to the memory dye channel is also shown. Comparison on the front view (XY) of the segmented cells and the CAAX image shows mostly accurate cell boundaries even under considerable “noise” in the membrane dye channel (due to membrane dye labeling endosomes from endocytosis during the membrane dye exposure) and correct cell-to-cell separation as best determinable by human experts.

Images 840, 850, and 860 show “side views” in the x-z dimension along cutline 811. The effectiveness of the model in achieving accurate membrane top even under the condition of low SNR in membrane dye image near top. The blue arrows point to some areas with very dim membrane signals. The red arrow points to an area with very low contrast (but not very dim). The green arrow points to an area where the membrane dye is significantly affected by endocytic vesicles. Zoomed-in images 870, 880, and 890 of the three cells (marked by I, II, III in the bottom image in panel B) are shown in panel C. The segmented boundary extracted from the cell instance segmentation is overlaid on the zoomed-in CAAX images. It can be seen that the segmented boundary is mostly along the center of the cell membrane marked by CAAX, which confirms the biological correctness of the cell segmentation.

After repeating the above qualitative inspection of cell and nuclear segmentation at the voxel level on about 20 randomly selected images, the inventors confirmed the segmented cells and nuclei demonstrated sufficient accuracy for the purpose of extracting all individual cells and nuclei in each field of view for further single-cell based quantitative analysis and modeling for the inventors' purposes. The inventors next evaluated the robustness of this segmentation accuracy over larger sized image sets at the instance level. The inventors assessed a set of 576 images from 22 different cell structure lines for the percentage of entire FOV's that contained successful segmentation in 3D via the DNA dye and membrane dye. Each image was manually scored by at least two human experts using an in-house scoring interface. The inventors found that over 98% of individual cells have successful nuclear and cell segmentation and over 80% of all images having the entire FOV successfully segmented.

### 5. Methods

5.1 Allen Cell Image Data Collection

The images in Allen Cell Data Collection were acquired in a microscopy imaging pipeline starting with gene-edited clonal hiPS cell lines with certain proteins (e.g., Lamin B1, H2B or CAAX) tagged with a fluorescence molecule (mEGFP for Lamin B1 or H2B and mTagRFPt for CAAX). Cells were plated on 96-well Matrigel-coated glass bottom plates and allowed 4-days of growth before imaging; detailed protocols can be found at www.allencell.org/methods-for-cells-in-the-lab.html. To illuminate the nucleus and cell membrane as positional references markers, the dyes NucBlue Live Ready Probe Reagent nucleus stain (excitation/emission 360/460 nm) and CellMask Deep Red Plasma membrane stain (excitation/emission 649/666 nm) were chosen, respectively, for of their compatibility with both mEGFP and mTagRFPt excitation/emission wavelengths. Dyes were added to the wells containing live cells and washed out after 30 min of incubation at 37° C., 5% CO2. Cells at selected positions were imaged on a Zeiss spinning-disk microscope with a Zeiss 100×/1.2 NA W C-Apochromat objective, a CSU-X1 Yokogawa spinning-disk head and Hammatsu Orca Flash 4.0 camera. Imaging in Z was performed from bottom to top with different excitation laser beams sequentially, resulting in multi-channel (protein of interest, membrane, nucleus, bright field) 3D images.

5.2 Semi-Automatic Technique for Obtaining Initial CAAX Segmentation

In some embodiments, the facility uses a seeded watershed algorithm for initial CAAX segmentation, using seeds obtained in a semi-automatic manner. First, the seeding model is applied (see Section 4.3) to generate one seed (a single connected component centered around the nucleus) inside each cell as the initial seeds. Then, the initial seeds are manually corrected in seeding model prediction images by (a) cutting the falsely merged seeds, (b) adding one seed manually (e.g., painting with one stroke in ImageJ) inside each cell that was only partly within the field-of-view and thus lacked automatically predicted seeds via the seeding model, (c) manually extending some seeds (less than 10%) where the cells had elongated extensions that the inventors needed to ensure the seeds were able to grow into by the watershed algorithm, and (d) adding an auxiliary bottom seed on the bottom slice and an auxiliary top seed on the top slice.

After the seeds are ready, the 3D microscopy image is upsampled to isotropic (from 0.108×0.108×0.29 um to 0.108×0.108×0.108 um) with cubic interpolation, and the seed image is upsampled accordingly with no interpolation.

Then, a 3D Gaussian smoothing is performed on the upsampled CAAX image and performed a seeded watershed on the smoothed image with the seeds that were semi-automatically generated above. In general, the watershed algorithm with no watershed line is more suitable for getting the thin membrane top, while the watershed algorithm with watershed line achieves more accurate separation boundary between cells. The watershed algorithm is then run twice, once without watershed line and the other with watershed line, both with a connectivity of 26 in 3D. The results are denoted as Wₗᵢₙₑ and W_{no_line}.

Finally, Wₗᵢₙₑ and W_{no_line} are merged into the CAAX segmentation.

Roughly speaking, Wₗᵢₙₑ and W_{no_line} are merged based on the watershed object from the top auxiliary seed in W_{no_line} (denoted by Ctop). Let B₁ be the watershed line in Wₗᵢₙₑ, which is equivalent to the cell membrane except the sub-optimal membrane top. Let B₂ be the boundaries of all cells in W_{no_line}, whose top parts can be viewed as a better estimation of the membrane top than B₁. The final CAAX segmentation is B₁ with voxels replaced by B₂ if less than 3 voxels away from Cₜₒₚ.

5.3 Training and Testing of the Deep Learning Models for Segmentation

For the nuclear segmentation model, cell membrane segmentation model, and the seeding model, the inventors used the Net_zoom model (with zoom ratio 3) in the inventors' Segmenter. Implementation details can be found at github.com/Allenlnstitute/aics-ml-segmentation, which is hereby incorporated by reference in its entirety. In some embodiments, training and testing are conducted on one or more NVIDIA Tesla V100 GPUs.

5.4 Deep Learning Model for Detecting Cell Pairs in Anaphase and Telophase

The inventors applied the cell and nucleus segmentation algorithm (without pair correction) on a manually selected subset of 60 images from the Allen Cell Image Data Collection where each image has at least one pair of cells in late anaphase or telophase/cytokinesis. The inventors created maximum intensity projections along Z on the segmentation results and calculate a 2D bounding box for each mitotic DNA pair based on their segmentation. The inventors then used these bounding boxes as the ground truth to train a 2D Faster-RCNN model to detect mitotic DNA pairs from the maximum intensity projections of the DNA dye image. The inventors used the off-the-shelf implementation from github.com/facebookresearch/maskrcnn-benchmark, which is hereby incorporated by reference in its entirety. The model was trained with learning rate 0.001.

### 6. Additional Applications

There are many other applications of the training assay approach. These include but are not limited to:

1. Structure segmentation from noisy images: In general, segmentation from images with lower signal to noise ratios (SNR) may be more accurate than segmentation from images with higher SNR. To improve the accuracy of the initial segmentation for iterative deep learning, in some embodiments, the training assay approach is applied by collecting a specialized biological experimental data. In some embodiments, the cells are fixed and images of identical cells collected twice, once with regular laser power appropriate for the primary image-based assay and one with a much higher laser power that would be detrimental to the primary assay but with which images can be generated with a higher SNR. The images acquired with the higher laser power can be used to generate preliminary ground truths for training a model to segment the target object from lower laser power images.

2. DNA and cell segmentation from transmitted light images: In some embodiments, the facility uses techniques described above to segment DNA and cells from transmitted light images, such as bright-field, instead of fluorescent images. In some embodiments, preliminary segmentation targets are obtained from a matched fluorescent image. The training assay approach thus provides an effective solution, which is similar to the solution described above, just using a different type of images as input to the deep learning model.

### 7. Conclusion

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method in a computing system for identifying 3D boundaries of each of a plurality of structures depicted in a 3D microscopy image of a distinguished scene captured using a first imaging technique, the method comprising:
accessing a plurality of 3D microscopy images each depicting a plurality of structures in a scene and each captured using a second imaging technique distinct from the first imaging technique;
using the accessed plurality of 3D microscopy images to train a first segmentation model for the second imaging technique;
for each of a plurality of scenes:
capturing the scene in a first 3D microscopy image using the first imaging technique;
capturing the scene in a second 3D microscopy image using the second imaging technique;
applying the first segmentation model to the second 3D microscopy image to obtain a segmentation for the scene;
assembling a training observation for the scene comprising the first 3D microscopy image and the obtained segmentation;
using the assembled training observations to train a second segmentation model for the first imaging technique;
imaging the distinguished scene using the first imaging technique to obtain the 3D microscopy image of the distinguished scene; and
subjecting the 3D microscopy image of the distinguished scene to the second segmentation model to obtain a segmentation specifying the 3D boundary of each of the plurality of structures depicted in the obtained 3D microscopy image of the distinguished scene.

2. The method of claim 1, wherein each of the plurality of scenes shows a biological sample.

3. The method of claim 1, further comprising storing the obtained segmentation specifying the 3D boundary of each of the plurality of structures depicted in the obtained 3D microscopy image of the distinguished scene.

4. The method of claim 1, further comprising causing a visual display to be presented that is at least in part based upon the obtained segmentation specifying the 3D boundary of each of the plurality of structures depicted in the obtained 3D microscopy image of the distinguished scene.

5. The method of claim 1 wherein the first imaging technique optically captures a DNA dye.

6. The method of claim 1 wherein the second imaging technique optically captures mEGFP-tagged Lamin B1.

7. The method of claim 1 wherein the first imaging technique optically captures a plasma membrane dye.

8. The method of claim 1 wherein the second imaging technique optically captures material edited to contain a CAAX protein.

9. One or more memories collectively having contents configured to cause a computing system to perform a method for segmenting a structure appearing in a distinguished image captured using a first image capture approach, the method comprising:
accessing a first segmentation model that outputs segmentations for images captured using a second image capture approach distinct from the first image capture approach;
for each of a plurality of scenes:
capturing the scene in a first image using the first imaging technique;
capturing the scene in a second image using the second imaging technique;
applying the accessed first segmentation model to the second image to obtain a segmentation for the scene;
assembling a training observation for the scene comprising the first image and the obtained segmentation;
using the assembled training observations to train a second segmentation model for the first imaging technique;
imaging the distinguished scene using the first imaging technique to obtain the image of the distinguished scene; and
subjecting the image of the distinguished scene to the second segmentation model to obtain a segmentation of the image of the distinguished scene.

10. The one or more memories of claim 9 wherein the captured images are 3D images.

11. The one or more memories of claim 9 wherein the captured images are 2D images.

12. The one or more memories of claim 9 wherein the captured images are captured using one or more microscope units.

13. The one or more memories of claim 9 wherein the captured images are captured using one or more microscope modalities.

14. The one or more memories of claim 9, the method further comprising:
training the first segmentation model.

15. One or more memories collectively storing a segmentation model data structure, the data structure comprising:
information comprising a machine learning model trained to predict segmentation for images captured in a first manner, the machine learning model having been trained using training observations each pairing an image of a scene captured in the first manner with a segmentation of an image of the same scene captured in a second manner distinct from the first manner, the segmentation of the image of the same scene captured in the second manner having been produced by applying to the image of the same scene captured in the second manner a model for segmenting images captured in the second manner,
such that the contents of the data structure can be applied to a distinguished image captured in the first manner to predict a segmentation of the distinguished image.

16. The one or more memories of claim 15 wherein the machine learning model is a neural network.

17. A method in a computing system for analyzing microscopy images of a distinguished sample of biological cells, the method comprising:
accessing a first image of the distinguished sample in which the distinguished sample has been subjected to DNA dye;
applying a first machine learning model to the first image to produce a segmentation of nuclei appearing in the first image;
accessing a second image of the distinguished sample in which the distinguished sample has been subjected to membrane dye;
applying a second machine learning model to the second image to produce a segmentation of cell bodies appearing in the first image; and
causing the produced segmentations of nuclei and cell bodies to be simultaneously displayed.

18. The method of claim 17 wherein the produced segmentations of nuclei and cell bodies are superimposed in their display.

19. The method of claim 17 wherein the produced segmentations of nuclei and cell bodies are displayed separately.

20. The method of claim 17, further comprising:
for each of a plurality of training samples of biological cells:
accessing a multi-channel microscopy image of the training sample of biological cells in which one channel is DNA dye and another channel is Lamin B1;
applying a third machine learning model to the Lamin B1 channel of the image to obtain a segmentation of nuclei for the image;
contributing the combination of (1) the segmentation of nuclei for the image and (2) the DNA dye channel to a training set; and
using the training set to train the first machine learning model.

21. The method of claim 17, further comprising:
for each of a plurality of training samples of biological cells:
accessing a multi-channel microscopy image of the training sample of biological cells in which one channel is DNA dye and another channel is Lamin B1;
applying a third machine learning model to the Lamin B1 channel of the image to obtain a segmentation of nuclei for the image;
contributing the combination of (1) the segmentation of nuclei for the image and (2) the DNA dye channel to a training set; and
using the training set to train the first machine learning model.

22. The method of claim 17, further comprising:
for each of a first plurality of training samples of biological cells:
accessing a multi-channel microscopy image of the training sample of biological cells in which one channel is DNA dye and another channel is Lamin B1;
applying a third machine learning model to the Lamin B1 channel of the image to obtain a Lamin B1 segmentation of nuclei for the image;
contributing the combination of (1) the Lamin B1 segmentation of nuclei for the image and (2) the DNA dye channel to a first training set;
using the first training set to train a fourth machine learning model for segmenting nuclei appearing in a DNA dye image;
for each of a second plurality of training samples of biological cells:
accessing a multi-channel microscopy image of the training sample of biological cells in which one channel is DNA dye and another channel is H2B;
applying the fourth machine learning model to the DNA dye channel of the image to obtain a DNA dye segmentation of nuclei for the image;
receiving input selecting interphase nuclei in the DNA dye segmentation;
applying a segmentation process to the H2B channel of the image to obtain a H2B segmentation of nuclei for the image;
receiving input selecting mitotic nuclei in the H2B segmentation;
merging the interphase nuclei selected in the DNA dye segmentation with the mitotic nuclei selected in the H2B segmentation to obtain a merged segmentation;
contributing the combination of (1) the merged segmentation of nuclei for the image and (2) the DNA dye channel to a second training set;
using the second training set to train the first machine learning model.

23. The method of claim 17, further comprising:
for each of a plurality of training samples of biological cells:
accessing a multi-channel microscopy image of the training sample of biological cells in which one channel is membrane dye and another channel is CAAX;
applying a third machine learning model to the CAAX channel of the image to obtain a segmentation of cell bodies for the image;
contributing the combination of (1) the segmentation of cell bodies for the image and (2) the membrane dye channel to a training set; and
using the training set to train the second machine learning model.

24. The method of claim 17, further comprising:
applying a seeding model to the first image to obtain a seeded nuclear segmentation in which adjacent nuclei are distinguished; and
assign differentiating indices to nuclei identified in the seeded nuclear segmentation,
and wherein the produced segmentation of nuclei are displayed in a manner that distinguishes nuclei to which different indices are assigned.

25. The method of claim 24, wherein the produced segmentation of cell bodies are displayed in a manner that distinguishes cell bodies containing nuclei to which different indices are assigned.

26. The method of claim 24, further comprising:
applying a pair detection model to the first image to obtain a pair detection result identifying divided pairs of nuclei; and
for each pair of nuclei identified by the pair detection result, causing both nuclei of the pair to be assigned the same index.
